(19) United States
(12) Patent Application Publication
(10) Pub. No.: US 2008/0294036 A1
(43) Pub. Date: Nov. 27, 2008

(54) Surgical Metal Detection Apparatus and Methods

(75) Inventors: Jennifer Hoi, Belmont, CA (US); Eli Luong, Garden Grove, CA (US); Laura Rupprecht, St. Paul, MN (US); Christine Medina, Pasadena, CA (US); David Huberdeau, Woodbridge, VA (US); Joe Chao, Sandinas, CA (US); Michelle Harran, Neptune City, NJ (US); Soumyadipta Acharya, Baltimore, MD (US)

Correspondence Address: PATENTBEST, 4600 ADELINE ST., #101, EMERYVILLE, CA, 94608, US

(73) Assignee: DEVICE EVOLUTIONS, LLC, Princeton, NJ (US)

(21) Appl. No.: 12/107,604

(22) Filed: Apr. 22, 2008

Related U.S. Application Data
(60) Provisional application No. 60/913,426, filed on Apr. 23, 2007.

(51) Int. Cl. A61B 5/00

(52) U.S. Cl. 600/424

(57) ABSTRACT

A surgical metal detection apparatus. The apparatus comprises an elongate, tubular cannula and a removable element, which may be a trocar, releasably engaged within the cannula. A metal detection coil is positioned within the removable element or, in some embodiments, within the cannula. The metal detection coil is connected to a metal detection circuit which is adapted to detect the presence of a metallic element proximate to the end of the removable element or cannula. When a metallic element has been detected, the removable element may be removed from the cannula to permit the passage of a removal tool, such as a screwdriver, through the cannula without removing or changing the position of the cannula.

### CROSS-REFERENCE TO RELATED APPLICATIONS

This application claims priority to, and the benefit of, U.S. Provisional Patent Application No. 60/913,426, filed Apr. 23, 2007. The contents of that application are incorporated by reference herein in their entirety.

### BACKGROUND OF THE INVENTION

1. Field of the Invention

The invention relates to surgical apparatus and methods and, more particularly, to surgical apparatus and methods for detecting surgical fasteners and other metallic elements.

2. Description of Related Art

Many surgical procedures require the temporary or permanent implantation of metallic fasteners. For example, in orthopedic surgical procedures, bone screws are often used to fix broken bones in place and to support bones while they heal. Pins, staples, and other metallic fasteners are also commonplace, and many of the clips, clamps, retractors, forceps, and other instruments used in the process of implanting them are also metallic.

There are certain situations in which it may be necessary or desirable to locate a metallic fastener or other element within the body. For example, currently, 5-10% of all implanted screws require removal due to complications like pain, infection, or ineffectiveness. Aside from complications, there are also situations in which it is desirable to remove a fastener after a bone or other structure has healed. Aside from fasteners and other elements that are deliberately implanted, metallic elements—like bullets, shrapnel, and other shards of metal—sometimes make their way into the body and require removal.

The location of the fastener or other metallic element may be difficult to visualize within the surgical field, because it may be obscured by blood or overgrown tissues. Moreover, in “minimally invasive” procedures in which the incision is under 3 cm, direct visualization of the fastener may not be possible. Even if the location of a fastener is known, it may still be difficult to remove it, because the surgeon generally must approach a fastener from a particular direction or use a particular angle of attack in order to engage and remove the fastener.

Fluoroscopy and ultrasound are two techniques that are often used to assist surgeons in locating metallic fasteners and other elements. Those techniques are certainly useful, but it can still be difficult to translate the two-dimensional information that fluoroscopy and ultrasound provide into a location within the three-dimensional volume of the body cavity. Even where fluoroscopy and ultrasound bring the surgeon to within a few millimeters of the fastener, problems may still arise in locating and engaging the fastener.

There have been some attempts to use metal detectors, either alone or as an adjunct to fluoroscopy and ultrasound, to detect fasteners within the body. For example, U.S. Pat. No. 2,393,717 to Speaker, which dates back to the 1940s, discloses basic circuitry for a surgical metal detector, and U.S. Pat. No. 4,526,177 to Rudy discloses a metal detector in the form of a plastic probe.

However, although a metal detector may provide useful information on the location of a metallic fastener or element within the body cavity, it may be difficult to coordinate the activity of the metal detector and the tool used to remove the fastener or element. Specifically, particularly if the incision is a small one, it may be difficult to insert the fastener removal tool without withdrawing or changing the position of the metal detector, which may cause the location of the fastener to be lost.

### SUMMARY OF THE INVENTION

One aspect of the invention relates to a surgical metal detection apparatus. The apparatus comprises an elongate, generally tubular cannula, an elongate removable element, a detection coil, and a detection circuit. The cannula has a first end, a second end, and a passageway between the first and second ends that is open at both ends. The removable element has first and second ends that correspond to the first and second ends of the cannula. The removable element is sized and adapted (1) to be removably retained in an engaged position substantially within the passageway of the cannula such that the second end of the removable element is proximate to the second end of the cannula, and (2) to be removed from the cannula to allow for the passage of a removal tool through the cannula. The detection coil is within the cannula or the removable element. The detection circuit is connected to the detection coil, and the detection circuit and detection coil are constructed and adapted to detect the presence of a metallic element proximate to the second end of the cannula or the second end of the removable element when the removable element is in its engaged position substantially within the passageway of the cannula.

Another aspect of the invention relates to a surgical metal detection apparatus. The apparatus comprises an elongate, generally tubular and substantially rigid cannula; an elongate trocar; and a detecting circuit. The trocar is adapted to fit removably within the passage of the cannula such that, when within the cannula a pointed end of the trocar lies just beyond and proximate to the second end of the cannula. The trocar has within it proximate to the pointed end at least a detecting element. The detecting circuit is connected to the detecting element within the trocar. The detecting circuit and detecting element are adapted to detect the presence of a metallic element proximate to the pointed end of the trocar.

Yet another aspect of the invention relates to a method of detecting and removing a metallic object during a surgical procedure. The method comprises inserting a metal detection probe within a cannula into a cavity. Upon an indication that a metallic object is proximate to the metal detection probe and cannula, the method further comprises removing the metal detection probe from the cannula while leaving the cannula in place within the cavity, inserting a removal tool into the cannula in the place of the metal detection probe, and removing the metallic object through the cannula using the removal tool.

Other aspects, features, and advantages of the invention will become apparent from the description that follows.

### BRIEF DESCRIPTION OF THE DRAWINGS

The invention will be described with respect to the following drawing figures, in which like reference numerals indicate like elements throughout the figures, and in which:

FIG. 1 is a perspective view of a surgical metal detection apparatus according to one embodiment of the invention;

FIG. 2 is an exploded perspective view of the surgical metal detection apparatus of FIG. 1;

FIG. 3 is a cross-sectional view of the surgical metal detection apparatus of FIG. 1, taken through Line 3-3 of FIG. 1;

FIG. 4 is a functional block diagram of the metal detection circuitry in the surgical metal detection apparatus of FIG. 1;

FIG. 5 is a partially sectional side elevational view illustrating the surgical metal detection apparatus of FIG. 1 in use during the process of locating a fastener;

FIG. 6 is a partially sectional side elevational view similar to the view of FIG. 5, illustrating the manner in which an outer cannula portion of the apparatus of FIG. 1 remains in position once a fastener has been located;

FIG. 7 is a partially sectional side elevational view similar to the view of FIG. 6, illustrating the removal of a fastener using the surgical metal detection apparatus of FIG. 1; and

FIG. 8 is a cross-sectional view of a surgical metal detector according to another embodiment of the invention, in which the positioning of the detecting components is different than in the surgical metal detector of FIG. 1.

### DETAILED DESCRIPTION

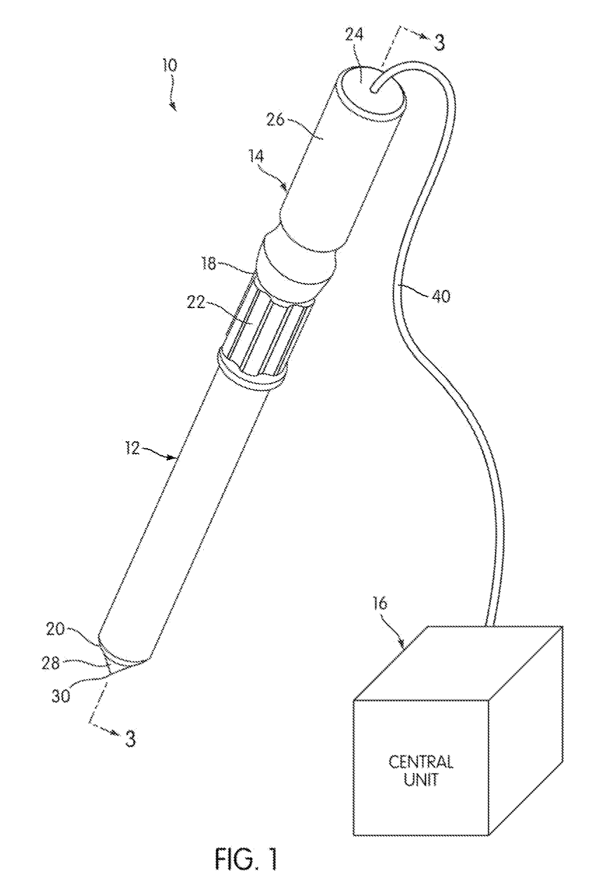

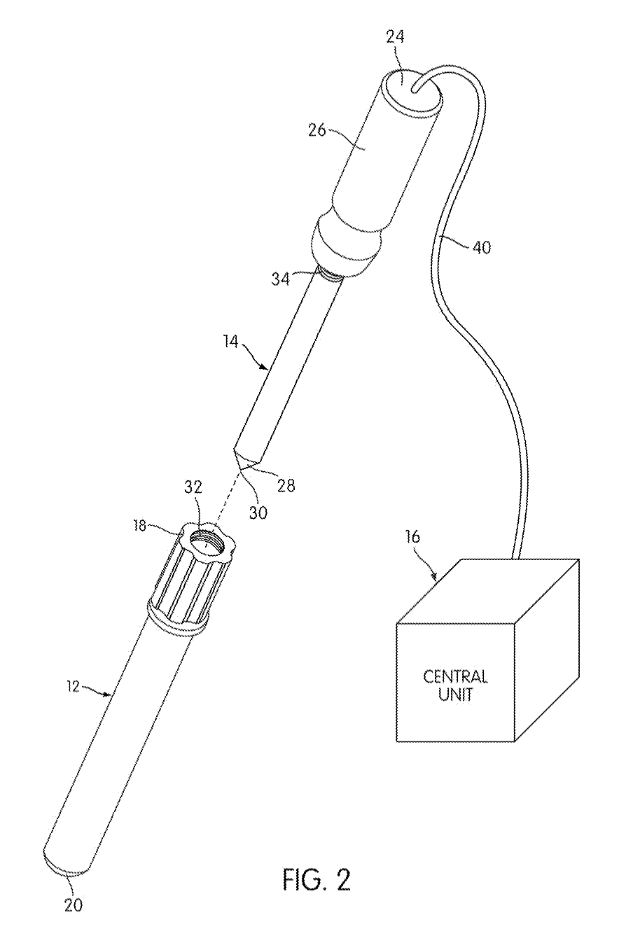

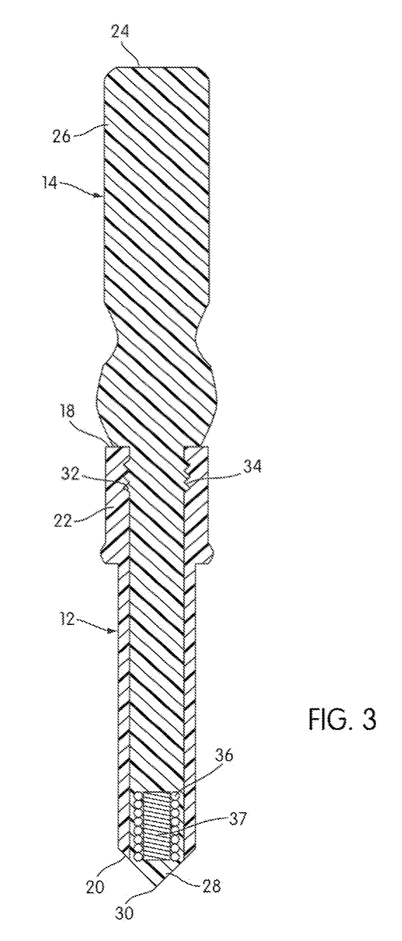

FIGS. 1-3 illustrate one embodiment of a surgical metal detection apparatus, generally indicated at 10. FIG. 1 is a perspective view of the surgical metal detection apparatus 10, FIG. 2 is an exploded perspective view of the surgical metal detection apparatus 10, and FIG. 3 is a cross-sectional view of the surgical metal detection apparatus 10, taken through Line 3-3 of FIG. 1.

The apparatus 10 comprises three main components: an elongate, generally tubular cannula 12, a removable element 14, and a central unit 16. The central unit 16 contains a metal detecting circuit and other support elements (not shown in FIGS. 1-3). Generally, at least the outer portions of the cannula 12 and the removable element 14 would be made of a biocompatible material, such as ultra high molecular weight polyethylene.

The cannula 12 has a first end 18 and a second end 20, and at least the second end 20 of the cannula 12 is adapted to be inserted into a body cavity to detect a metallic fastener or element that lies proximate to the second end 20. Depending on the size of the body cavity and the particular application for which it is intended, the cannula 12 may thus have a variety of sizes and diameters. In most embodiments, it is advantageous if the cannula 12 is at least somewhat rigid.

Toward its first end 18, the cannula 12 flares into a grip portion 22 that is adapted to allow the user to grip the cannula 12. Toward its second end 20, the edge of the cannula 12 may be tapered, beveled, or angled, as will be described in more detail below.

The removable element 14 is elongate, generally rodlike, and is adapted to fit and be removably retained within the cannula 12. It has a first end 24 with a handle portion 26 that lies adjacent to the first end 18 of the cannula 12 when the removable element 14 is fully engaged in the cannula 12, and a second end 28 that is adapted for insertion into a body cavity along with the second end 20 of the cannula 12.

The dimensions of the removable element 14 and the cannula 12 may vary from embodiment to embodiment, but in one embodiment, for example, the cannula 12 may have an overall length of approximately 90 mm, provide an internal lumen with a diameter of approximately 6 mm, and have a wall thickness of approximately 2 mm, for an overall outer diameter of approximately 10 mm. The removable element 14 may have a diameter of approximately 6 mm and a length selected such that when in the fully engaged position, the second end 28 of the removable element 14 substantially coincides with the second end 20 of the cannula. In general, it may be desirable to size the cannula 12 so that it can fit through an incision size of no greater than about 3 cm, so that the apparatus can be used in so-called “minimally invasive” surgery.

As was noted above, in the illustrated embodiment, the removable element 14 is a trocar, a surgical penetrating tool. A trocar provides the apparatus 10 with the ability to penetrate tissue in search of a metallic fastener or element, a capability that may be useful in some embodiments, but that may not be necessary and is not required in all embodiments.

In embodiments in which the removable element 14 is a trocar, the second end 28 of the removable element 14 has a point or other structure suitable for penetrating tissue, as is the case in FIGS. 1-3. If the second end 28 has penetrating structure other than a point, that structure may have any suitable characteristics. As shown in FIGS. 1-3, the removable element 14 has a conical point 30 that makes a 40° angle. Trocars with sharper or more steeply angled points may have more penetrating power. However, a sharper or more steeply angled point may increase the length of removable element 14 that protrudes beyond the second end 20 of the cannula 12 when the cannula 12 and removable element 14 are engaged, and it may be desirable to limit the length of the removable element 14 that extends beyond the second end 20 of the cannula 12, so that the apparatus 10 can be positioned immediately proximate to a metallic fastener or element when one is detected. For that reason, it may be desirable to use a removable element 14 with a shallower point angle. If the removable element 14 is a trocar, then the taper in the second end 20 of the cannula 12 may match the angle of the trocar point 30, as it does in apparatus 10.

The removable element 14 or other removable element 14 is retained within the cannula 12 by means of cooperating engaging structures on the cannula 12 and removable element 14. In the apparatus 10, sets of screw threads 32, 34, one set 32 on the interior sidewall of the cannula 12 proximate to its first end 18, and a corresponding set of screw threads 34 on the removable element 14 just below the handle portion 26 secure the cannula 12 and removable element 14 in engagement. Clips, tabs, slots, and other similar structures may also be used. In other embodiments, the removable element 14 may be retained within the cannula 12 without specific engaging structures; for example, the two components 12, 14 may be held together by a tight fit.

As is best shown in the cross-sectional view of FIG. 3, near its second end 28, the removable element 14 of the apparatus 10 carries a detection coil 36. The detection coil 36 is comprised of ferromagnetic wire, which, in apparatus 10, is wrapped around a ferromagnetic core 37. The detection coil 36 forms a part of a metal detection circuit, which will be described below in more detail. When energized, the detection coil 36 generates a magnetic field that is used by the metal detection circuit to detect the presence of a metallic fastener or element. The number of turns in the coil and the corresponding inductance value of the coil 36 may vary from embodiment to embodiment. The coil 36 may be comprised, for example, of 30-gauge wire wrapped tightly so that the coil 36 has an overall length of approximately 23.5 mm and an overall diameter of approximately 4 mm. (For simplicity in illustration, only a few turns of the coil 36 are shown in FIG. 3.)

The manner in which the coil 36 is contained and held in place within the removable element 14 may vary. For example, if the removable element 14 is formed of a moldable plastic, such as ultra high molecular weight polyethylene, the coil 36 could be placed in an injection mold and the plastic of which the removable element 14 is comprised could be injection molded around it. If the removable element 14 is to be machined, a coil compartment of a suitable size and location could be created by boring out the center of the removable element 14 from the first end 24 or by boring or milling a suitable compartment for the coil 36 through the sidewall of the removable element 14. If the coil 36 is situated in a machined compartment, any excess space in that compartment could be filled by a plug of appropriate size and characteristics so as to hold the coil 36 in place and prevent it from shifting. More generally, any method of placing and securing the coil 36 within the removable element 14 may be used so long as the coil 36 remains in position once placed.

The coil 36 is connected by a wire 40 to the external central unit 16. In some embodiments, the wire 40 may be continuous; in other embodiments, plugs or jacks may be provided so that the coil 36 and removable element 14 can be disconnected from the central unit 16. The wire 40 may attach to any point on the removable element 14 at or near its first end 24, although it may be advantageous to select a point of attachment that minimizes wire strain. (For ease in illustration, the wire 40 is not shown in FIG. 3)

The central unit 16 contains circuitry and any other components that are used in performing the metal detecting function of the apparatus 10. As those of skill in the art will realize, in some embodiments, some or all of the components that will be described below as being contained in the central unit 16 may be made internal to the removable element 14 or the cannula 12. In those embodiments, a separate, external central unit 16 may not be necessary.

The description that follows focuses on the circuitry and methods by which the apparatus 10 is adapted to detect metallic fasteners and other elements. In the following description, it will be assumed that the apparatus 10 uses the beat frequency oscillation technique for metal detection. However, other metal detection methods (and appropriate corresponding circuits) may be used if desired.

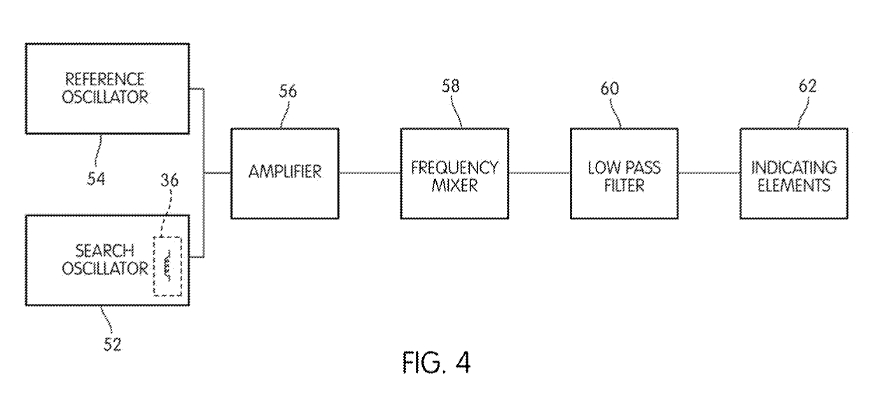

FIG. 4 is a functional block diagram of a beat frequency oscillation detection circuit, generally indicated at 50. The detection circuit 50 has two oscillators, a search oscillator 52 and a reference oscillator 54. The coil 36 within the removable element 14 is an inductor and is a part of the search oscillator 52. (The coil 36 may have an inductance of, for example, about 60 μH.) The other components of the search oscillator 52 include the resistors and capacitors that cause the search oscillator 52 to oscillate with a characteristic frequency. The characteristic frequency of the search oscillator 52 may vary from embodiment to embodiment; however, 540 kHz has been found to be an appropriate frequency in at least some embodiments.

The reference oscillator 54 may be comprised of essentially the same circuit as the search oscillator 54, with the exception that, instead of the coil 36, an inductor of equivalent inductance would be provided. (Thus, although equivalent in terms of function, the reference oscillator 54 may be a physically smaller circuit located entirely within the central unit 16.) The reference oscillator 54 may be set to have the same characteristic frequency as the search oscillator 52. Alternatively, in some embodiments, the frequency of the search oscillator 52 and the frequency of the reference oscillator 54 may be very slightly offset, for example, by about 100 Hz.

Although making the reference oscillator 54 circuit essentially the same as the search oscillator 54 circuit may be expedient, the reference oscillator 54 may be essentially any type of component that can be made to oscillate at a characteristic frequency. For example, in some embodiments, a piezoelectric crystal may be used as an oscillator.

The signals from the search oscillator 52 and the reference oscillator 54 are passed through one or more amplifiers 56 before being input into a frequency mixer 58. For purposes of illustration, FIG. 4 shows a single amplifier 56 that amplifies the signals from both the search oscillator 52 and the reference oscillator 54. That may be the case in some embodiments, although in other embodiments, an amplifier 56 may be provided for or as a part of each of the oscillator circuits 52, 54. The output from the frequency mixer 58, which is the difference between the two frequencies, is passed through a low-pass filter 60, and the output from the low-pass filter 60 is used to drive an indicating element 62, such as a speaker, a light or set of lights, or a vibrator, to name a few.

When the coil 36 within the removable element 14 is brought near a metallic fastener or element, the magnetic field around the coil 36 changes, which causes the characteristic frequency of the search oscillator 52, of which the coil 36 is a part, to shift. The closer the coil 36 is to the fastener or element, the more the frequency shifts. The frequency mixer 58 outputs the difference in frequency between the search oscillator 52 and the reference oscillator 54, and the low-pass filter 60 is set with a cut-off frequency sufficient to filter out any unwanted high-frequency components. The cut-off frequency of the low-pass filter 60 may be just less than the sum of the characteristic frequencies of the search oscillator 52 and the reference oscillator 54. For example, if the search oscillator 52 and the reference oscillator 54 are set to oscillate at 540 kHz, the cut-off frequency of the low-pass filter 60 may be set to 1,000 kHz.

If the indicating element 62 is a speaker, when the removable element 14 is brought near a metallic fastener or element, the user will hear a noise, and that noise will increase in frequency as the removable element 14 is brought closer to the object. (If the characteristic frequencies of the search oscillator 52 and the reference oscillator 54 are slightly offset, as was described above, then the user would hear a noise with a frequency equal to the offset, e.g., 100 Hz, and that noise would change in frequency as the coil 36 neared the metallic fastener or element.) The useful range of the apparatus 10 will depend on the characteristics of the coil 36 and the elements within the detection circuit 50, among other factors. An apparatus 10 that can detect a fastener within 2-10 mm of the second end 20 of the cannula 12 is suitable. Embodiments in which the apparatus within at least about 4 mm of the second end 20 of the cannula may be particularly useful.

It is also possible to change the sensitivity of the apparatus 10 so that the detection range is decreased. This can be done by adjusting the characteristic oscillating frequency of the search oscillator 52 or the reference oscillator 54. Generally speaking, the closer the characteristic oscillating frequencies of the search oscillator 52 and the reference oscillator 54 are, the closer a metallic fastener or element must be to the coil 36 before a discernible frequency offset is created. Many circuits include, for example, variable capacitors or inductors, and these may be used to change the characteristic frequency, and with it, the detection range of the apparatus 10.

Any indication that the removable element 14 is near a metallic fastener or object may be used: the indicating element 62 may be a light that flashes when a fastener or object is detected, it may be a vibrator that vibrates more intensely as the removable element 14 nears the fastener or element, or it may be a visual indication, such as a waveform on an oscilloscope, to name a few types of indicating elements 62. Several indicating elements may be used in combination, so that the user hears and sees indications that the fastener or element is near. As those of skill in the art will realize, additional components may be necessary to adapt the output from the low-pass filter 60 to drive some types of indicating elements 62.

It may be advantageous if the frequencies of the oscillators 52, 54, the filter 60 and the other components are set such that when the apparatus 10 is near metal, the user receives a continuous indication or signal to that effect, for example, a continuous sound that changes in frequency as the user draws closer. By contrast, if the user is provided with only a beat or pulse indication as he or she passes near the fastener, it is possible that the user could miss that beat or pulse.

The detection circuit 50 may be powered by direct current (DC), for example, by a 9V DC power supply. In some embodiments, the central unit 16 may include a transformer for converting standard alternating current (AC) power to 9V DC power. In other embodiments, power may be provided by a battery, or by a conventional wired power supply and a battery in combination. Some elements of the detection circuit 50, such as the low-pass filter 60, may be provided with their own power supplies, or with a direct connection to the main power supply.

Circuits for performing metal detection by beat frequency oscillation are known in the art, and appropriate circuit diagrams can be obtained from many sources. For example, suitable oscillator circuits are disclosed in Rakes, Charles D., “Basic Circuitry: Metal Detection,” Poptronics, August, 2001, and Rakes, Charles D., “Basic Circuitry: Metal Detection II,” Poptronics, September, 2001. Those two articles are incorporated by reference in their entireties, and the latter article in particular discloses appropriate oscillator circuitry for the search and reference oscillators 52, 54. Additionally, U.S. Pat. No. 4,526,177, which was incorporated by reference above, describes a metal detection circuit using a piezoelectric crystal reference oscillator, and that circuit may be used in some embodiments of the invention.

In other embodiments of the invention, very low frequency (VLF) and pulse induction (PI) methods of metal detection may be used. All of these technologies are capable of producing an indication that a metallic fastener or object is near; the difference between them lies in what is measured to make that determination (e.g., voltage, frequency, fall time, etc.).

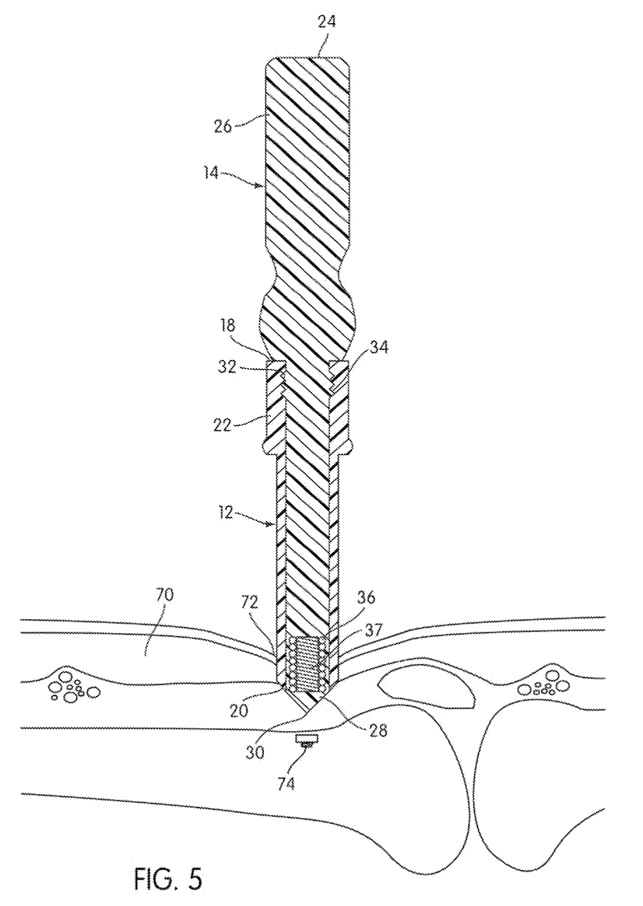

FIGS. 5-7 are partially sectional side elevational views illustrating the process of using the apparatus 10 to find a fastener. In FIG. 5, the apparatus 10 has been inserted into a body cavity 70 through a small incision 72 and the user, presumably a surgeon, sweeps the cavity with the combined apparatus 10 until the fastener 74 has been located and is directly beneath the second end 28 of the removable element 14. During this operation, even if the removable element 14 is not used to penetrate tissue per se, it may be useful in deflecting tissue and easing the passage of the cannula 12 into the body cavity 70. In FIG. 5, the position and size of the fastener 74 are slightly exaggerated for clarity and ease in illustration; in actual use, the fastener 74 may be deeply embedded in bone and covered by other structures, making it difficult or impossible to visualize.

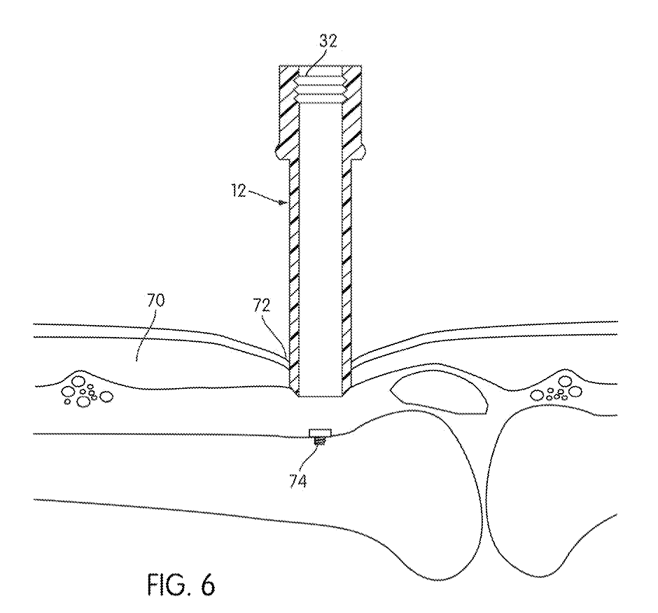

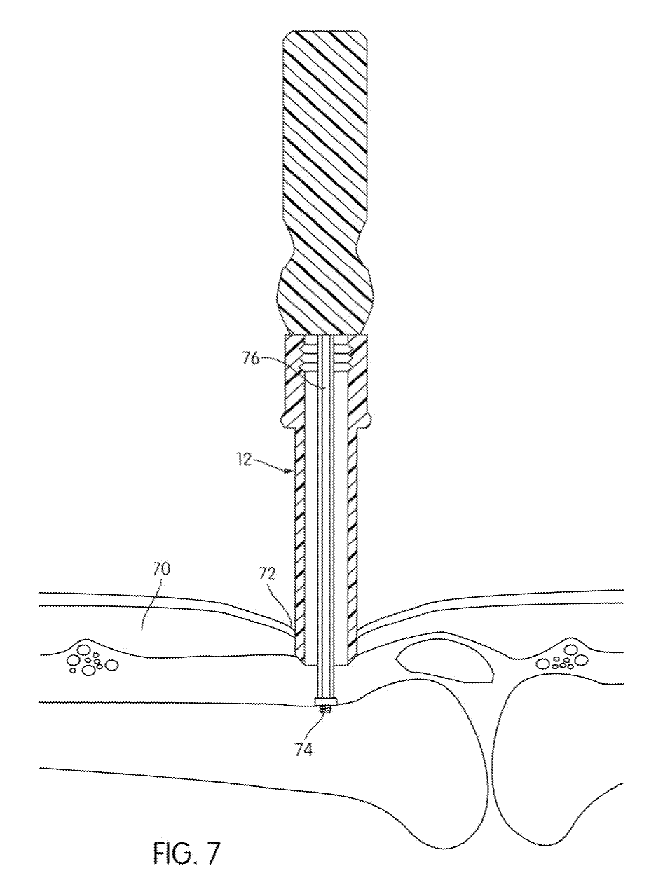

As shown in FIG. 6, once the fastener 74 has been located, the user unscrews and removes the removable element 14. Then, as shown in FIG. 7, while holding the cannula 12 in place, the user inserts a removal tool 76 into the cannula 12 to remove the fastener. Thus, the cannula 12 maintains its position and ensures that the fastener 74 is not lost again during the process of inserting a tool to remove it. Once the fastener has been removed, both the tool 76 and the cannula 12 are removed.

Depending on the particular type of fastener, several types of removal tools 76 may need to be used to remove a single fastener 74. For example, a driver tool may be used to loosen the fastener 74, and then the driver tool may be replaced by a grasper to allow the fastener 74 to be removed from the body cavity 70 through the cannula 12. If necessary or desirable, fluoroscopy, ultrasound, or another type of visualization technique may be used in conjunction with the apparatus 10; preferably, the material of which the cannula 12 and removable element 14 are made is radio opaque or otherwise visible when using such techniques.

The cannula 12 may also be used as an access point for other surgical tools. Once used, the apparatus 10 may be chemically sterilized for re-use or, in some embodiments, at least the cannula 12 and removable element 14 components of the apparatus 10 may be disposable.

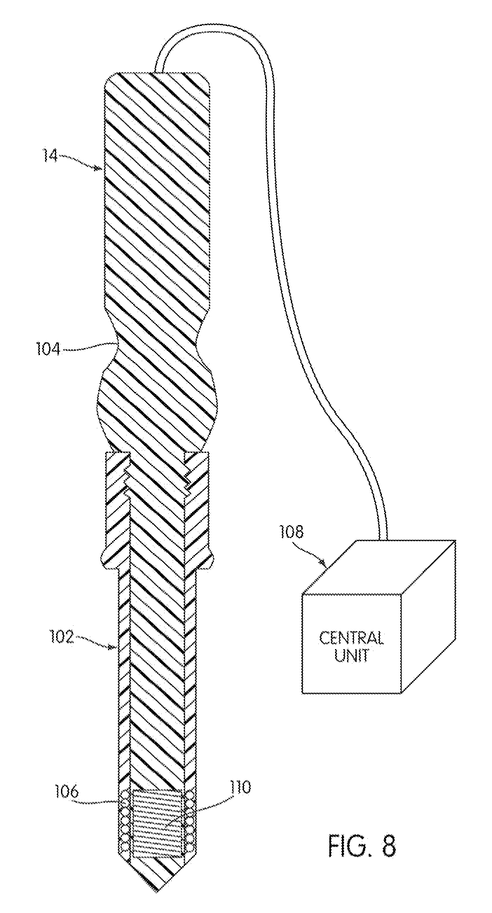

FIG. 8 is a cross-sectional view illustrating another embodiment of surgical metal detection apparatus, generally indicated at 100. The apparatus 100 is substantially similar to the apparatus 10; therefore, any components not described here may be assumed to be the same or essentially the same as those described above with respect to the apparatus 10.

The apparatus 100 has a cannula 102 and a removable element 104, which, as shown in FIG. 8, comprises a trocar. The primary difference between the apparatus 100 and the apparatus 10 is that in the apparatus 100, a detection coil 106 is provided within the cannula 102, instead of within the trocar 104, and it is the cannula that connects with the central unit 108.

In the apparatus 100, a ferrite element 110 is provided in a position such that when the cannula 102 and trocar 104 are engaged, the ferrite element 110 acts as a ferrite core for the detection coil 106 in the cannula. However, in some embodiments, if the magnetic field produced by the detection coil 106 is strong enough to perform its detecting function without a core, the trocar 104 may not include a ferrite element 110. Additionally, although ferrite cores have been disclosed in this description, those of skill in the art will realize that any material with an appropriate magnetic permittivity constant may be used in embodiments of the invention.

In one advantageous embodiment, the removable element 104 may be a metallic removal tool, such as a screwdriver or an Allen key, instead of a trocar.

As those of skill in the art will realize, it may be helpful to calibrate the apparatus 10, 100 from time to time, particularly if variable or adjustable sensitivities are used. For that purpose, the apparatus 10, 100 may be supplied with a calibration stand that holds the apparatus 10, 100 at a specified, known distance from a metal block.

Although the invention has been described with respect to certain exemplary embodiments, the description is intended to be illuminating, rather than limiting. Various modifications and changes may be made within the scope of the invention, which is determined by the claims.

What is claimed is:

1. A surgical metal detection apparatus, comprising:
an elongate, generally tubular cannula having a first end, a second end opposite the first end, and a passageway between the first and second ends that is open at both ends;
an elongate removable element having first and second ends corresponding to the first and second ends of the cannula, the removable element being sized and adapted (1) to be removably retained in an engaged position substantially within the passageway of the cannula such that the second end of the removable element is proximate to the second end of the cannula, and (2) to be removed from the cannula to allow for the passage of a removal tool through the cannula;
a detection coil within the cannula or the removable element; and
a detection circuit connected to the detection coil, the detection circuit and detection coil being constructed and adapted to detect the presence of a metallic element proximate to the second end of the cannula or the second end of the removable element when the removable element is in its engaged position substantially within the passageway of the cannula.

2. The surgical metal detection apparatus of claim 1, wherein the cannula is substantially rigid.

3. The surgical metal detection apparatus of claim 1, wherein the cannula and the removable element are formed of biocompatible materials.

4. The surgical metal detection apparatus of claim 3, wherein the biocompatible materials are plastics.

5. The surgical metal detection apparatus of claim 1, wherein the detection circuit is a beat frequency oscillation metal detection circuit.

6. The surgical metal detection apparatus of claim 1, wherein the removable element is a trocar.

7. The surgical metal detection apparatus of claim 1, wherein the cannula and the removable element have cooperating engaging structures constructed and arranged to retain the removable element in the engaged position.

8. The surgical metal detection apparatus of claim 7, wherein the cooperating engaging structures comprise a set of threads on an interior surface of the cannula and a cooperating set of threads on an exterior surface of the removable element.

9. The surgical metal detection apparatus of claim 1, wherein the detection coil is within the removable element proximate to the second end of the removable element.

10. The surgical metal detection apparatus of claim 9, wherein the detection coil includes a ferrite core.

11. The surgical metal detection apparatus of claim 1, wherein the detection coil is within the cannula proximate to the second end of the cannula.

12. The surgical metal detection apparatus of claim 11, wherein the removable element includes a ferrite element proximate to its second end positioned such that when the removable element is in the engaged position, the ferrite element acts as a ferrite core for the detection coil within the cannula.

13. The surgical metal detection apparatus of claim 1, the detection circuit further comprising one or more indicating elements adapted to indicate the presence of the metallic element to a user.

14. The surgical metal detection apparatus of claim 13, wherein the one or more indicating elements are selected from the group consisting of lights, speakers, and vibrational elements.

15. A surgical metal detection apparatus, comprising:
an elongate, generally tubular and substantially rigid cannula having a first end, a second end opposite the first end, and a passageway between the first and second ends that is open at both ends; and
an elongate trocar constructed and adapted to fit removably within the passageway of the cannula such that, when within the cannula, a pointed end of the trocar lies just beyond and proximate to the second end of the cannula, the trocar having within it proximate to the pointed end at least a detecting element; and
a detecting circuit connected to the detecting element within the trocar, the detecting circuit and detecting element being adapted to detect the presence of a metallic element proximate to the pointed end of the trocar.

16. The surgical metal detection apparatus of claim 15, wherein the detecting circuit comprises a reference oscillator adapted to oscillate at a first defined frequency and a search oscillator adapted to oscillate at a second frequency.

17. The surgical metal detection apparatus of claim 16, wherein the detecting element comprises a portion of the search oscillator that, in the presence of a metallic object, causes the second frequency to change.

18. A method of detecting and removing a metallic object during a surgical procedure, comprising:
inserting a metal detection probe within a cannula into a cavity; and
upon an indication that a metallic object is proximate to the metal detection probe and cannula,
removing the metal detection probe from the cannula while leaving the cannula in place within the cavity,
inserting a removal tool into the cannula in the place of the metal detection probe, and
removing the metallic object through the cannula using the removal tool.

19. The method of claim 18, wherein the metal detection probe has the form of a trocar with a pointed end.

20. The method of claim 18, wherein the indication that a metallic object is proximate to the metal detection probe comprises a substantially continuous indication that changes as the object becomes nearer.
